(19) United States
(12) Patent Application Publication
(10) Pub. No.: US 2010/0249243 A1
(43) Pub. Date: Sep. 30, 2010

(54) NOVEL BETA-HYDROXYKETONES AND BETA-ALKOXYKETONES WITH ESTROGENIC ACTIVITY

(76) Inventors: Juha Pulkkinen, Kuopio (FI); Paavo Honkakoski, Kuopio (FI); Mikael Peräkylä, Kuopio (FI); Istvan Berczi, Lorette (CA); Reino Laatikainen, Kuopio (FI)

Correspondence Address: BIRCH STEWART KOLASCH & BIRCH, PO BOX 747, FALLS CHURCH, VA, 22040-0747, US

(21) Appl. No.: 12/744,072

(22) PCT Filed: Nov. 21, 2008
(86) PCT No.: PCT/FI08/50674

§ 371 Date: May 21, 2010

(30) Foreign Application Priority Data
Nov. 23, 2007 (FI) 2007-0898

(51) Int. Cl. A61K 31/12; C07C 49/82; C07C 49/84; C07C 49/175; A61P 19/00; A61P 3/06; A61P 9/00; A61P 3/04; A61P 25/22; A61P 25/28; A61P 25/24; A61P 29/00; A61P 15/00; A61P 9/12; A61P 27/02; A61P 35/00

(52) U.S. Cl. 514/676; 568/306; 568/331; 514/685; 568/328; 514/682; 568/325; 514/679

(57) ABSTRACT

This invention relates to β-hydroxyketones and β-alkoxyketones of formula (I), to their use as estrogen receptor modulators, and to methods for their preparation.

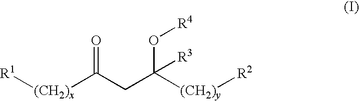

### FIELD OF THE INVENTION

This invention relates to certain β-hydroxyketones and β-alkoxyketones, to their use as estrogen receptor modulators, and to methods for their preparation.

### BACKGROUND OF THE INVENTION

The nuclear hormone receptor superfamily is a very important target for drug development. Members of this group include estrogen, androgen, progesterone, and glucocorticoid receptors, the activity of which can be controlled by ligand binding, as well as constitutive androstane and pregnane X receptors which have an essential role in drug metabolism. Nuclear hormone receptors regulate expression of their target genes, which control the essential metabolic reactions and differentiation processes of cells. Thus, nuclear receptor disorders are of great clinical importance.

Naturally occurring and synthetic estrogens have been widely utilized in the treatment of variety of disorders including cardiovascular disease, menopausal symptoms, dysmenorrhea, acne, prostatic cancer, hirsutism, osteoporosis and hot flashes. On the other hand, estrogen antagonists can be used in the treatment of diseases or conditions such as breast cancer, osteoporosis and anovulation. Because of this huge therapeutic value, there is a continuous need for artificial compounds which mimic estrogen-like behaviour in such a way that they have selective effects on different estrogen responsive tissues (selective estrogen receptor modulators i.e. SERMs). Also, it should be possible to utilize such compounds without the negative side effects of the estrogen replacement therapy. There are two subtypes of estrogen receptors: ERα and ERβ. Both forms bind to and are activated by their common natural ligand 3,17β-estradiol (E2), and none of the ER agonists or antagonists currently in clinical use are specific for either form. Because of the serious adverse effects of ER agonists and antagonists, great variation in ERα and β expression in diverse target tissues, and cell- and promoter-specific functions displayed by the ER subtypes, there are increasing efforts to explore new chemical scaffolds to develop both subtype-specific and tissue selective ligands.

Various methods for preparing β-hydroxyketones have been disclosed for example in a) Tetrahedron Lett. 1997, 38(44), 7705-7708, b) J. Org. Chem. 1991, 56(3), 1325-1327, c) J. Chem. Res. Miniprint 1988, 2520-2547, d) Chem. Ber. 1976, 109, 640-649.

Further methods for preparing β-hydroxyketones and β-methoxyketones are disclosed in publications e) Chem. Lett. 1976, 559-562, f) J. Chem. Soc. Perkin Trans. 1 1981, 82-87, g) Tetrahedron Lett. 1999, 40(52), 9371-9374, h) J. Am. Chem. Soc. 1999, 121(17), 4168-4178, i) J. Am. Chem. Soc. 1983, 195(6), 1664-5, j) Helv. Chim. Acta 1998, 81(2), 251-267, k) J. Org. Chem. 1983, 48(18), 2957-2962, l) Eur. J. Med. Chem. 1992, 27(7), 663-672, m) Chem. Lett. 1977, 153-154, n) Chem. Asian J. 2006, 1-2, 210-215, o) Indian J. Chem. 2007, 45B, 1355-1358, p) Beijing Shifan Daxue Xuebao, Ziran Kexueban 2004, 40, 92-96, q) Angew. Chem. Int. Ed. 2006, 45(48), 8200-8203, r) J. Am. Chem. Soc. 2004, 126(43), 13942-13944, s) PCT Int. Appl. 2005, WO2005012209 A2, t) Chem. Lett. 1995, 987-988, u) Bull. Chem. Soc. Jpn. 1985, 58(11), 3385-3386, and v) Chem. Lett. 1974, 15-16.

However, the above mentioned publications do not suggest any pharmaceutical use for the disclosed compounds.

In conclusion, there is a need for small non-steroidal molecules which can act as agonists or antagonists for nuclear hormone receptors such as ERα and ERβ. We now describe a novel set of such compounds having estrogen activity in vitro and a further group of such compounds which are now for the first time disclosed to have estrogen activity in vitro.

### DESCRIPTION OF THE INVENTION

This invention provides novel compounds of the formula (I)

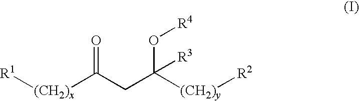

or stereoisomers, pharmaceutically acceptable salts or prodrug forms thereof, wherein
x is an integer from 0 to 3 and y is an integer from 0 to 2, provided that x and y do not have the same value;
R¹ and R² are the same or different phenyl or naphthyl groups of the formula

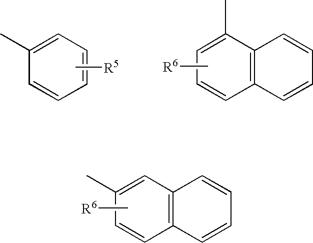

and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl;
provided that
when the compound has the formula (I) and x is 0, y is 1, R³ is hydrogen and R⁴ is hydrogen, then R¹ cannot be an unsubstituted phenyl,^{a} 2-hydroxyphenyl^{b} or 3-methoxyphenyl^{c} if R² is unsubstituted phenyl, and then R¹ cannot be 4-chlorophenyl if R² also is 4-chlorophenyl^{d} [a)-d)], and
when the compound has the formula (I) and x is 0, y is 2, R³ is hydrogen and R⁴ is hydrogen, then R¹ cannot be an unsubstituted phenyl,^{e} 2-hydroxyphenyl,^{b} 4-hydroxyphenyl,^{f} 4-methoxyphenyl,^{g} 3-nitrophenyl^{h} or 4-chlorophenyl^{g} if R² is an unsubstituted phenyl, [e)-h)], and
when the compound has the formula (I) and x is 1, y is 0, R³ is hydrogen and R⁴ is hydrogen, then R¹ cannot be an unsubstituted phenylⁱ or 3-fluorophenyl^{j} if R² is un-substituted phenyl, and then R¹ cannot be an unsubstituted phenyl^{k} or 3,4-dimethoxyphenyl^{l} if R² is 4-methoxyphenyl, and then R¹ cannot be 2-chlorophenyl if R² is 2-chlorophenyl^{d}, [i)-1)] and
when the compound has the formula (I) and x is 2, y is 0, and R⁴ is hydrogen, then R³ cannot be hydrogen if R¹ is an unsubstituted phenyl and R² is an unsubstituted phenyl^{m} or 4-nitrophenl^{n, o}, or if R¹ is 4-hydroxy-3-methoxyphenyl and R² is an un-substituted phenyl^{p} or 4-hydroxy-3-methoxyphenyl^{p}, and then R³ cannot be methyl, if R¹ and R² are simultaneously unsubstituted phenyls^{q}, [m)-q)] and
when the compound has the formula (I) and x is 2, y is 1, R³ is hydrogen and R⁴ is hydrogen, then R¹ cannot be an unsubstituted phenyl if R² also is an unsubstituted phenyl^{r} [r)], and
when the compound has formula (I) and x is 3, y is 0, R³ is hydrogen^{s} or methyl^{t} and R⁴ is hydrogen, then R¹ cannot be an unsubstituted phenyl if R² also is an unsubstituted phenyl, [s)-t)] and
when the compound has the formula (I) and x is 0, y is 1^{u} or 2^{v}, R³ is hydrogen and R⁴ is methyl, then R¹ cannot be an unsubstituted phenyl if R² also is an unsubstituted phenyl [u)-v)].

In the compounds of formula (I), integer x is preferably 2 or 3, but it may also be 0 or 1. Integer y in the compounds of formula (I) is preferably 0 or 1, but it may also be 2. However, integers y and x do not have the same value.

R¹ and R² may independently of each other be unsubstituted or ortho-, meta- or para-substituted by 0-5 substituent groups R⁵ or R⁶. Preferably groups R¹ and R² are independently of each other unsubstituted or substituted by one substituent R⁵ or R⁶.

In the preferred compounds of formula (I), if groups R¹ and R² are substituted, preferred substituents R⁵ and R⁶ are selected from the group consisting of hydrogen, lower alkyl, lower alkoxyl, halogen, nitro and hydroxyl. Alkyl or alkoxyl groups may be further substituted by the above mentioned groups.

Even more preferably substituents R⁵ and R⁶ are lower alkoxy, halogen or hydroxyl.

In the preferred compounds of formula (I), substituent R³ is hydrogen or a C₁₋₄ alkyl, especially methyl.

In the preferred compounds of formula (I), substituent R⁴ is hydrogen or methyl.

In the preferred compounds of formula (I), R¹ and R² are independently an unsubstituted phenyl or phenyl monosubstituted with alkoxy, halogen or hydroxyl.

In the context of the present application, the general terms used above and below preferably have the following meanings:

A compound having estrogen activity means a compound which acts as an agonist, antagonist, partial agonist or inverse agonist for nuclear hormone receptors such as ERα and ERβ.

A prodrug is a drug which is administered in an inactive or significantly less active form but once administered, it is metabolised in vivo into the active compound.

Alkyl is a saturated hydrocarbon radical containing 1-20, preferably 1-8 carbon atoms. It is for example methyl, ethyl, n-propyl, isopropyl, n-butyl, isobutyl, sec-butyl, tert-butyl, pentyl, hexyl, heptyl or octyl.

Lower alkyl contains 1-6, preferably 1-4 carbon atoms. It is for example ethyl, methyl, n-propyl, isopropyl, n-butyl, isobutyl, sec-butyl, tert-butyl, pentyl or hexyl, preferably methyl, ethyl, n-propyl or isopropyl.

In this description lower alkoxy contains 1-6, preferably 1-2 carbon atoms. It is for example methoxy, ethoxy, n-propoxy, isopropoxy, n-butoxy, isobutoxy, sec-butoxy, tert-butoxy, pentyloxy or ethyloxy, preferably methoxy.

Halogen is chlorine, bromine, fluorine or iodine, preferably fluorine or chlorine.

The compounds of the invention may contain one or more asymmetric centers and can thus occur as racemates and racemic mixtures, single enantiomers, diastereomeric mixtures and individual diastereomers. The present invention is meant to comprehend all such isomeric forms of the compounds of the invention.

The invention relates particularly to the compounds of formula (I)

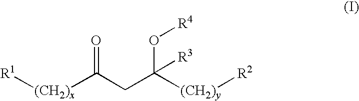

or stereoisomers, pharmaceutically acceptable salts or prodrug forms thereof, wherein x is 2 or 3 and y is 0 or 1, R¹ and R² are both phenyl groups which are independently substituted by one substituent selected from the group consisting of alkoxy, halogen or hydroxyl, R³ is hydrogen or methyl and R⁴ is hydrogen or methyl.

Preferred examples of the compounds of the invention are selected from the group consisting of 5-hydroxy-1-(4-hydroxy-phenyl)-5-phenyl-hexan-3-one (23), 5-hydroxy-6-(4-hydroxy-phenyl)-1-phenyl-hexan-3-one (25), 5-hydroxy-1-(4-hydroxy-phenyl)-6-phenyl-hexan-3-one (31), 1-hydroxy-6-(4-hydroxy-phenyl)-1-phenyl-hexan-3-one (34), 1-hydroxy-1-(3-hydroxy-phenyl)-6-(4-hydroxy-phenyl)-hexan-3-one (35), 6-hydroxy-1,6-diphenyl-heptan-4-one (36), 6-hydroxy-1-(4-hydroxy-phenyl)-6-phenyl-heptan-4-one (37), 2-hydroxy-7-(4-hydroxy-phenyl)-1-phenyl-heptan-4-one (40), 5-methoxy-6-(4-fluorophenyl)-1-phenyl-hexan-3-one (48), 5-methoxy-6-naphthalen-2-yl-1-phenyl-hexan-3-one (50), 2-methoxy-1,7-diphenyl-heptan-4-one (51), and stereoisomers, pharmaceutically acceptable salts and prodrug forms thereof.

The invention also relates to pharmaceutical compositions which contain a compound of the formula (I) or a stereoisomer, pharmaceutically acceptable salt or prodrug form thereof as active ingredient. These pharmaceutical compositions are for example those for enteral, such as in particular oral, those for parenteral administration, and those for local administration to warm-blooded animals, in particular to humans.

The pharmaceutical compositions according to the invention usually contain the pharmacologically active ingredient according to formula (I) together with known pharmaceutical excipients. The amount of the active ingredient in the pharmaceutical compositions according to the invention is, for example, from about 0.001% to 100% by weight, preferably from about 0.1% to about 50% by weight. The dose of the active ingredient can depend on various factors, such as the efficacy of the active ingredient, severity of the disease to be treated or its symptoms, administration procedure, sex, age, weight and/or individual condition of the subject in need of the treatment. In a normal case, for a human adult of about 75 kg in weight, one daily dose of about 1 mg to about 1000 mg, in particular from about 10 mg to about 500 mg, is to be estimated. This can be administered as a single dose or in several sub-doses.

The invention also relates to the use of the compounds of the formula (I) or stereoisomers, pharmaceutically acceptable salts or prodrug forms thereof for the preparation of pharmaceutical compositions for the treatment of disease states, disorders and conditions alleviated by compounds having estrogen activity. Among such conditions may be mentioned bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

The invention also provides the compounds of formula (I) or stereoisomers, pharmaceutically acceptable salts or prodrug forms thereof for use in a method for treating disease states, disorders or conditions alleviated by compounds having estrogen activity.

The invention also provides a method for the therapeutic or prophylactic treatment of disease states, disorders and conditions alleviated by compounds having estrogen activity, said method comprising administering an effective amount of a compound of formula (I) to a subject in need of such treatment.

A still further object of the invention is a method for the therapeutic or prophylactic treatment of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate, said method comprising administering an effective amount of a compound according to the invention to a subject in need of such treatment.

The compounds of the formula (I) can be prepared as described below. The reagents used in the preparation of the compounds of this invention can be either commercially obtained or can be prepared by standard procedures described in the literature.

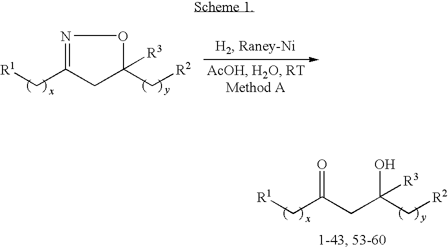

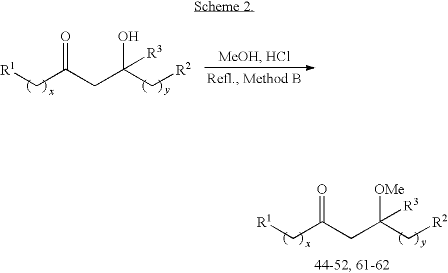

A process for preparing the compounds of formula (I) thus comprises the general steps of catalytic hydrogenation of appropriate 4,5-dihydroisoxazoles in the presence of Raney-Ni, acetic acid and water to afford the desired β-hydroxyketones (compounds 1-43 and 53-60) according to Scheme 1, or methylation of the corresponding β-hydroxyketones in the presence of methanol and hydrochloric acid to afford the desired β-methoxyketones (compounds 44-52 and 61-62) according to Scheme 2.

The invention also relates to the compounds of formula (I)

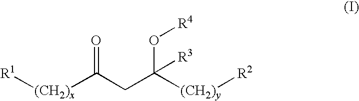

or stereoisomers, pharmaceutically acceptable salts or prodrug forms thereof, wherein
x is an integer from 0 to 3 and y is an integer from 0 to 2, provided that x and y do not have the same value;

R¹ and R² are the same or different phenyl or naphthyl groups of the formula

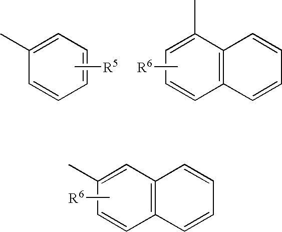

and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl,
for use as pharmaceuticals, as well as for use in a method for treating disease states, disorders or conditions alleviated by compounds having estrogen activity.

Further objects of the invention are
a pharmaceutical composition comprising a compound of the formula (I)

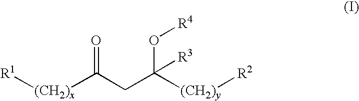

or a stereoisomer, pharmaceutically acceptable salt or a prodrug form thereof, wherein
x is an integer from 0 to 3 and y is an integer from 0 to 2, provided that x and y do not have the same value;
R¹ and R² are the same or different phenyl or naphthyl groups of the formula

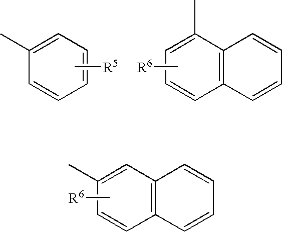

and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl, in association with a pharmaceutically acceptable carrier, and
the use of said compounds for the manufacture of medicaments for the treatment of disease states, disorders or conditions alleviated by compounds having estrogen activity.

The invention also provides a method for treating disease states, disorders or conditions alleviated by compounds having estrogen activity, said method comprising administering an effective amount of a compound of formula (I)

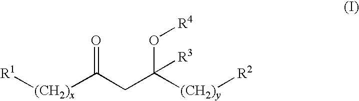

or a stereoisomer, pharmaceutically acceptable salt or a prodrug form thereof, wherein
x is an integer from 0 to 3 and y is an integer from 0 to 2, provided that x and y do not have the same value;
R¹ and R² are the same or different phenyl or naphthyl groups of the formula

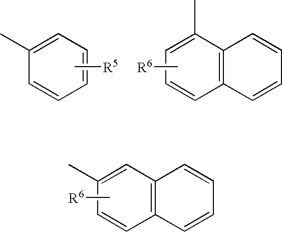

and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl, to a subject in need of such treatment.

The following examples further illustrate the invention described above.

### EXAMPLES 1-43

### 3-Hydroxy-1-(2-nitrophenyl)-4-phenyl-butan-1-one (1)

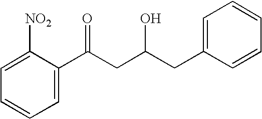

Method A: A solution of 5-Benzyl-3-(2-nitrophenyl)-4,5-dihydroisoxazole (2.11 g, 0.0074 mol), acetic acid (4.42 g, 0.074 mol) and water (13.3 ml, 0.74 mol), in 40 ml of 1:1 methanol-tetrahydrofurane is stirred under an atmospheric pressure of H₂ at room temperature overnight in the presence of Raney nickel (1.3 g). The catalyst is removed by filtering through Celite and the filtrate extracted with dichloromethane. The extracts are washed with saturated NaHCO₃, dried with MgSO₄ and evaporated to dryness.

Yield 68%, mp=93.0-96.0° C., ¹H NMR (CDCl₃): δ 7.36-7.22 (m, 5H), 7.14 (ddd, 1H, J=7.7, 7.7, 1.4), 7.09 (dd, 1H, J=7.9, 1.3), 6.71 (dd, 1H, J=8.1, 0.5), 6.67 (ddd, 1H, J=7.8, 7.8, 0.9), 5.46 (br s, 1H, OH), 4.87 (m, 1H), 3.38 (dd, 1H, J=16.3, 10.1), 3.18-3.09 (m, 2H), 2.86 (dd, 1H, J=13.8, 7.1); ¹³C NMR δ 158.1, 146.6, 137.1 (3 s), 130.6, 129.5, 129.3, 128.7, 126.7, 116.5, 115.8 (7 d), 111.8 (s), 80.1 (d), 40.9, 40.9 (2 t).

The following compounds included in the invention were prepared by Method A using appropriate starting materials:

### 4-(3,4-Dimethoxyphenyl)-3-hydroxy-1-phenyl-butan-1-one (2)

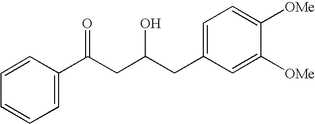

Yield 32%, mp=79.5-80.8° C., ¹H NMR (CDCl₃): δ 7.91 (m, 2H), 7.57 (m, 1H), 7.45 (m, 2H), 6.84-6.75 (m, 3H), 4.46 (m, 1H), 3.86 (s, 3H), 3.85 (s, 3H), 3.23 (br d, 1H, OH, J=3.5), 3.14 (dd, 1H, J=17.5, 3.5), 3.08 (dd, 1H, J=17.5, 8.2), 2.89 (dd, 1H, J=13.7, 7.2), 2.80 (dd, 1H, J=13.7, 6.1); ¹³C NMR δ 200.2, 148.9, 147.3, 137.0, 134.3 (5 s), 133.0, 128.6, 128.0, 120.3, 111.8, 111.2, (6 d), 55.9, 55.8 (2 q), 37.6, 34.8, 25.9 (3 t).

### 3-Hydroxy-4-(4-methoxyphenyl)-1-(2-nitrophenyl)-butan-1-one (3)

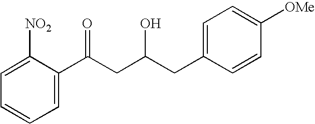

Yield 73%, mp=92.0-95.0° C., ¹H NMR (CDCl₃): δ 7.17 (m, 2H), 7.14 (ddd, 1H, J=8.3, 7.2, 1.5), 7.09 (dd, 1H, J=7.9, 1.4), 6.86 (m, 2H), 6.71 (dd, 1H, J=8.2, 0.9), 6.67 (ddd, 1H, J=7.9, 7.2, 1.2), 5.65 (br s, 1H, OH), 4.82 (m, 1H), 3.78 (s, 3H), 3.38 (dd, 1H, J=16.2, 10.1), 3.12 (dd, 1H, J=16.2, 7.8), 3.08 (dd, 1H, J=14.0, 6.3), 2.81 (dd, 1H, J=14.0, 7.1); ¹³C NMR δ 198.9, 157.9, 139.4, 135.3, 133.6 (5 s), 129.4, 129.5, 128.9, 113.8 (4 d), 55.3, (q), 37.6, 34.2, 25.8 (3 t).

### 3-Hydroxy-1-(2-methoxyphenyl)-4-phenylbutan-1-one (4)

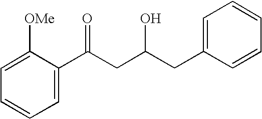

Yield 32%, pale yellow oil, ¹H NMR (CDCl₃): δ 7.72 (dd, 1H, J=7.7, 1.8), 7.46 (ddd, 1H, J=7.3, 6.6, 1.8), 7.34-7.20 (m, 5H), 6.96 (ddd, 1H, J=7.7, 7.2, 0.9), 6.93 (d, 1H, J=8.4), 4.41 (m, 1H), 3.80 (m, 3H), 3.30 (s, 1H, OH), 3.27 (dd, 1H, J=17.8, 2.8), 3.04 (dd, 1H, J=17.8, 9.0), 2.94 (dd, 1H, J=13.5, 6.9), 2.80 (dd, 1H, J=13.5, 6.5); ¹³C NMR δ 202.6, 158.9, 138.4 (3 s), 134.1, 130.3, 129.5, 128.4 (4 d), 127.7 (s), 126.4, 120.7, 111.6, 69.3 (4 d), 55.4 (q), 49.5, 43.1 (2 t).

### 3-Hydroxy-1-(2-methoxyphenyl)-4-(4-methoxyphenyl)-butan-1-one (5)

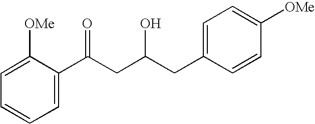

Yield 34%, yellow oil, ¹H NMR (CDCl₃): δ 7.71 (dd, 1H, J=7.7, 1.8), 7.46 (ddd, 1 H, J=8.5, 7.4, 1.8), 7.17 (m, 2H), 6.99 (ddd, 1H, J=7.7, 7.7, 0.8), 6.94 (d, 1H, J=8.4), 6.85 (m, 2H), 4.36 (m, 1H), 3.83 (s, 3H), 3.79 (s, 3H), 3.26 (d, OH, 1H, J=3.3), 3.25 (dd, 1H, J=17.8, 2.8), 3.05 (dd, 1H, J=17.8, 9.0), 2.87 (dd, 1H, J=13.6, 6.9), 2.75 (dd, 1H, J=13.6, 6.4); ¹³C NMR δ 202.6, 158.9, 158.2 (3 s), 134.0, 130.5 (2 d), 130.3, 127.7 (2 s), 120.7, 113.8, 111.6, 69.4 (4 d), 55.4, 55.3 (2 q), 49.5, 42.1 (2 t).

### 3-Hydroxy-1-(4-methoxyphenyl)-4-phenyl-butan-1-one (6)

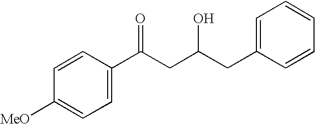

Yield 65%, mp=74.5-75.0° C., ¹H NMR (CDCl₃): δ 7.88 (m, 2H), 7.34-7.20 (m, 5H), 6.90 (m, 2H), 4.46 (m, 1H), 3.84 (s, 3H), 3.41 (d, 1H, OH, J=3.3), 3.09 (dd, 1H, J=17.4, 3.2), 3.00 (dd, 1H, J=17.4, 8.5), 2.96 (dd, 1H, J=13.6, 7.0), 2.83 (dd, 1H, J=13.6, 6.4); ¹³C NMR δ 199.1, 163.8, 138.2 (3 s), 130.4 (d), 129.8 (s), 129.5, 128.5, 126.5, 113.8, 69.1 (5 d), 55.5 (q), 43.6, 43.0 (2 t).

### 3-Hydroxy-1-(4-methoxyphenyl)-4-(2-methoxyphenyl)-butan-1-one (7)

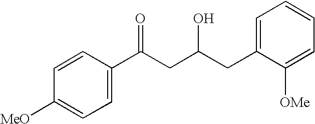

Yield 46%, a colourless wax; ¹H NMR δ 7.88 (m, 2H), 7.20 (m, 2H), 6.92-6.86 (m, 3H), 6.85 (d, 1H, J=8.0), 4.48 (m, 1H), 3.85 (s, 3H), 3.78 (s, 3H), 3.41 (d, 1H, OH, J=3.8), 3.07 (dd, 1H, J=17.2, 4.0), 3.04 (dd, 1H, J=17.2, 7.7), 2.97 (dd, 1H, J=13.4, 6.7), 2.93 (dd, 1H, J=13.4, 6.4); ¹³C NMR δ 199.2, 163.7, 157.6 (3 s), 131.5, 130.4 (2 d), 130.1 (s), 127.9 (d), 126.5 (s), 120.6, 113.7, 110.4, 68.3 (4 d), 55.5, 55.3 (2 q), 43.8, 37.5 (2 t).

### 1,4-Di-(4-methoxyphenyl)-3-hydroxybutan-1-one (8)

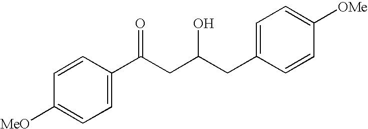

Yield 61%, mp=93.0-95.6° C., ¹H NMR (CDCl₃): δ 7.88 (m, 2H), 7.17 (m, 2H), 6.91 (m, 2H), 6.85 (m, 2H), 4.42 (m, 1H), 3.85 (s, 2H), 3.78 (s, 3H), 3.39 (b s, 1H, OH), 3.09 (dd, 1H, J=17.3, 3.1), 2.99 (dd, 1H, J=17.3, 8.6), 2.90 (dd, 1H, J=13.7, 6.8), 2.78 (dd, 1H, J=13.7, 6.4); ¹³C NMR δ 199.2, 163.8, 158.4 (3 s), 130.4, 130.4 (2 d), 129.9, 129.4 (2 s), 113.9, 113.8, 69.2 (3 d), 55.5, 55.2 (2 q), 43.6, 42.0 (2 t).

### 4-(3,4-Dimethoxyphenyl)-3-hydroxy-1-(4-methoxyphenyl)-butan-1-one (9)

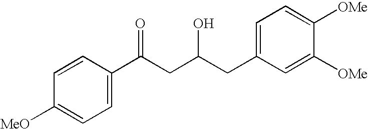

Yield 67%, pale brown viscous oil, ¹HNMR (CDCl₃): δ 7.89 (m, 2H), 6.91 (m, 2H), 6.84-6.72 (m, 3H), 4.44 (m, 1H), 3.87 (s, 3H), 3.85 (s, 3H), 3.86, (s 3H), 3.42 (br s, 1H, OH), 3.11 (dd, 1H, J=17.3, 3.3), 3.02 (dd, 1H, J=17.3, 8.4), 2.89 (dd, 1H, J=13.7, 7.1), 2.80 (dd, 1H, J=13.7, 6.2); ¹³C NMR δ 199.1, 163.9, 148.9, 147.7, 130.7 (5 s), 130.4 (d), 129.9 (s), 121.4, 113.8, 112.6, 111.3, 69.2 (5 d), 55.9, 55.8, 55.5 (3 q), 43.6, 42.5 (2 t).

### 4-(4-Fluorophenyl)-3-hydroxy-1-(4-methoxyphenyl)-butan-1-one (10)

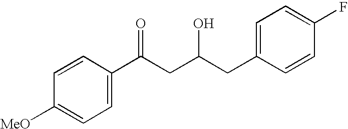

Yield 46%, mp=99.5-100.5° C., ¹H NMR δ 7.95 (m, 2H), 7.29 (m, 2H), 7.06 (m, 2 H), 6.99 (m, 2H), 4.49 (m, 1H), 3.93 (s, 3H), 3.47 (d, 1H, OH, J=2.8), 3.15 (dd, 1H, J=17.3, 3.1), 3.07 (dd, 1H, J=17.3, 8.6), 2.97 (dd, 1H, J=13.8, 7.3), 2.87 (dd, 1H, J=13.8, 5.9); ¹³C NMR δ 199.0, 163.9 (2 s), 161.7, 133.9 (2 d), 130.9 (dd), 130.4 (d), 129.6 (s), 115.2 (dd), 113.9 (s), 69.0 (d), 55.5 (q), 43.6, 42.1 (2 t).

### 3-Hydroxy-1-(4-methoxyphenyl)-4-pentafluorophenyl-butan-1-one (11)

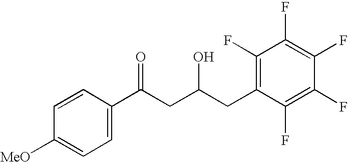

Yield 31%, mp=103.8-104.3° C., ¹H NMR δ 7.98 (m, 2H), 7.01 (m, 2H), 4.68 (m, 1H), 3.95 (s, 3H), 3.65 (d, 1H, OH, J=3.8), 3.24 (dd, 1H, J=17.5, 2.7), 3.16 (dd, 1H, J=17.5, 8.9), 3.10 (dd, 1H, J=13.9, 7.9), 2.98 (dd, 1H, J=13.9, 5.2); ¹³C NMR δ 198.5, 164.2 (2 s), 145.5, 140.0, 137.5 (3 d), 130.5 (d), 129.6 (s), 114.0 (d), 111.8 (s), 67.1 (d), 55.6 (q), 43.6, 29.3 (2 t).

### 4-[4-(2-Dimethylamino-ethoxy)phenyl]-3-hydroxy-1-(4-methoxy-phenyl)-butan-1-one (12)

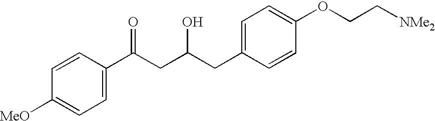

Yield 65%, yellow viscous oil, ¹H NMR δ 7.88 (m, 2H), 7.15 (m, 2H), 6.90 (m, 2H), 6.87 (m, 2H), 4.39 (m, 1H), 4.04 (t, 2H, J=5.8), 3.85 (s, 3H), 3.07 (dd, 1H, J=17.2, 3.1), 2.99 (dd, 1H, J=17.2, 8.6), 2.88 (dd, 1H, J=13.6, 7.1), 2.77 (dd, 1H, J=13.6, 6.3), 2.71 (t, 2H, J=5.8), 2.32 (s, 6H); ¹³C NMR δ 199.1, 163.8, 157.6 (2 s), 130.4, 130.4 (2 d), 130.3, 129.9 (2 s), 114.6, 113.8, 69.2 (3 d), 66.0, 58.3 (2 t), 55.5, 45.9 (2 q), 43.6, 42.1 (2 t).

### 3-hydroxy-4-(1-methoxy-naphthalen-2-yl)-1-(4-methoxyphenyl)-butan-1-one (13)

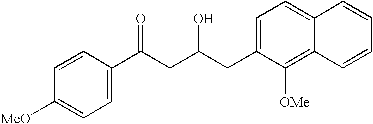

Yield 19%, brown viscous oil, ¹H NMR δ 8.17 (d, 1H, J=8.4), 7.95 (m, 2H), 7.90 (d, 1H, J=8.0), 7.67 (d, 1H, J=8.4), 7.59 (m, 1H), 7.53 (m, 1H), 7.48 (d, 2H, J=8.4), 6.96 (m, 2H), 4.67 (m, 1H), 4.03 (s, 3H), 3.91 (s, 3H), 3.69 (d, 1H, J=3.1), 3.27-3.14 (m, 4H); ¹³C NMR δ 199.2, 163.8, 154.1, 134.2 (4 s), 130.4 (d), 130.0 (s), 128.9, 128.0 (2 d), 126.5 (s), 126.0, 125.7, 124.2, 122.1, 113.8, 68.9 (6 d), 61.9, 55.5 (2 q), 43.8, 36.9 (2 t).

### 1-(4-methoxyphenyl)-4-(3-methoxy-naphthalen-2-yl)-3-hydroxy-1-butanone (14)

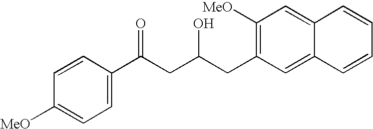

Yield 29%, brown viscous oil, ¹H NMR δ 8.09 (d, 1H, J=8.6), 7.80 (m, 2H) 7.74 (d, 1H, J=8.2), 7.72 (d, 1H, J=9.0), 7.46 (m, 1H), 7.30 (m, 1H), 7.22 (d, 1H, J=9.0), 6.83 (m, 2H), 4.56 (m, 1H), 3.84 (s, 3H), 3.77 (s, 3H), 3.65 (d, 1H, OH J=3.8), 3.46 (dd, 1H, J=13.6, 7.3), 3.42 (dd, 1H, J=13.6, 6.5), 3.13 (dd, 1H, J=17.4, 7.5), 3.05 (dd, 1H, J=17.4, 3.8); ¹³C NMR δ 199.3, 163.6, 155.0, 133.6 (4 s), 130.3 (d), 130.1, 129.3 (2 s), 128.5, 126.6, 123.6, 123.4 (4 d), 119.5 (s), 113.7, 113.0, 68.8 (3 d), 56.3, 55.4 (2 q), 43.7, 32.0 (2 t).

### 1-(4-Chlorophenyl)-3-hydroxy-4-phenyl-butan-1-one (15)

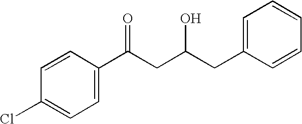

Yield 32%, mp=71.0-72.8° C., ¹H NMR (CDCl₃): δ 7.82 (m, 2H), 7.40 (m, 2H), 7.34-7.21 (m, 5H), 4.47 (m, 1H), 3.15 (br s, OH, 1H), 3.12-3.01 (m, 2H), 2.94 (dd, 1 H, J=13.6, 7.1), 2.83 (dd, 1H, J=13.6, 6.3); ¹³C NMR δ 199.1, 140.0, 137.9, 135.1 (4 s), 129.5, 129.4, 129.0, 128.6, 126.6, 68.8 (6 d), 44.2, 43.0 (2 t).

### 1-(4-Chlorophenyl)-3-hydroxy-4-(4-methoxyphenyl)-butan-1-one (16)

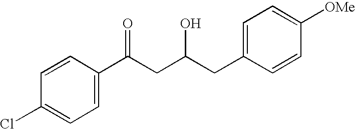

Yield 47%, mp=88.0-90.0° C., ¹H NMR (CDCl₃): δ 7.83 (m, 2H), 7.41 (m, 2H), 7.16 (m, 2H), 6.85 (m, 2H), 4.42 (m, 1H), 3.78 (s, 3H), 3.10 (d, OH, 1H, J=3.6), 3.08 (dd, 1H, J=17.5, 3.9), 3.03 (dd, 1H, J=17.5, 7.9), 2.88 (dd, 1H, J=13.7, 7.1), 2.78 (dd, 1H, J=13.7, 6.2); ¹³C NMR δ 199.2, 158.4, 139.9, 135.1 (4 s), 130.4 (d), 129.9 (s), 129.5, 129.0, 114.0, 69.0 (4 d), 55.2 (q), 44.1, 42.1 (2 t).

### 1-(4-Chlorophenyl)-4-(3,4-dimethoxyphenyl)-3-hydroxy-butan-1-one (17)

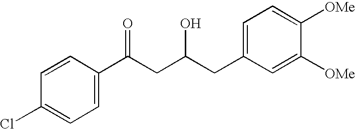

Yield 56%, pale brown viscous oil, ¹H NMR (CDCl₃): δ 7.84 (m, 2H), 7.42 (m, 2H), 7.83-6.70 (m, 3H), 4.46 (m, 1H), 3.11 (br s, 1H, OH), 3.08-3.03 (m, 2H), 2.89 (dd, 1H, J=13.7, 7.2), 2.79 (dd, 1H, J=13.7, 6.1); ¹³C NMR δ 199.2, 148.9, 147.8, 140.0, 135.1, 130.4 (6 s), 129.5, 129.0, 121.4, 112.5, 111.3, 69.0 (6 d), 55.9, 55.9 (2 q), 44.2, 42.6 (2 t).

### 3-Hydroxy-1-(4-hydroxyphenyl)-4-phenyl-butan-1-one (18)

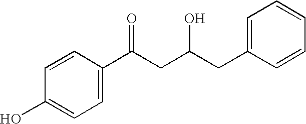

Yield 49%, a dark brown oil, ¹H NMR δ 9.00 (br s, 1H, OH), 7.79 (m, 2H), 7.31 (m, 2H), 7.27-7.21 (m, 3H), 6.81 (m, 2H), 4.46 (m, 1H), 3.61 (br s, 1H, OH), 3.07 (dd, 1H, J=17.3, 3.1), 3.00 (dd, 1H, J=17.3, 8.6), 2.96 (dd, 1H, J=13.6, 7.0), 2.84 (dd, 1H, J=13.6, 6.5); ¹³C NMR δ 199.4, 161.8, 138.0 (3 s), 130.8, 129.5, 129.1 (3 d), 129.1 (s), 128.7, 126.6, 115.4, 71.6 (4 d), 43.4, 42.9 (2 t).

### 4-Hydroxy-4-(3-hydroxyphenyl)-1-phenyl-butan-2-one (19)

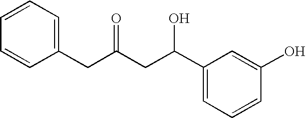

Yield 63%, a pale yellow oil, ¹H NMR δ 7.28-7.22 (m, 2H), 7.20 (m, 1H), 7.07 (m, 3H), 6.74 (m, 1H), 6.71 (d, 1H, J=7.7), 6.65 (d, 1H, J=7.7), 5.00 (dd, 1H, J=9.2, 3.3), 3.61 (d, 1H, J=15.7), 3.61 (d, 1H, J=15.7), 2.82 (dd, 1H, J=17.2, 9.2), 2.69 (dd, 1H, J=17.3, 3.3); ¹³C NMR δ 209.1, 156.2, 144.1, 133.3 (4 s), 129.8, 129.5, 128.8, 127.2, 117.6, 115.0, 112.8, 70.0 (8 d), 50.7, 50.0 (2 t).

### 4-Hydroxy-1,6-diphenylhexan-2-one (20)

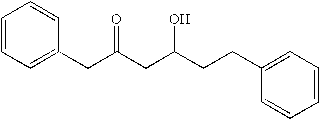

Yield 91%, a yellow wax; ¹H NMR δ 7.35-7.28 (m, 2H), 7.26-7.21 (m, 3H), 7.17-7.12 (m, 5H), 4.00 (m, 1H), 3.65 (s, 2H), 3.01 (br s, 1H, OH), 2.75 (m, 1H), 2.65-2.52 (m, 3H), 1.75 (m, 1H), 1.63 (m, 1H); ¹³C NMR δ 209.3, 141.8, 133.6 (3 s), 129.4, 128.8, 128.4, 128.4, 127.2, 125.8, 66.9 (7 d), 50.7, 48.4, 38.0, 31.7 (4 t).

### 1-(3-Fluorophenyl)-4-hydroxy-6-phenyl-hexan-2-one (21)

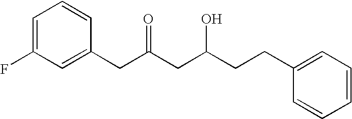

Yield 66%, a colourless viscous oil; ¹H NMR (CDCl₃): δ 7.30-7.23 (m, 3H), 7.18-7.15 (m, 3H), 6.99-6.87 (m, 3H), 4.03 (m, 1H), 3.68 (s, 2H), 2.93 (br s, 1H, OH), 2.76 (m, 1H), 2.65 (m, 1H), 2.60 (m, 2H), 1.79 (m, 1H), 1.67 (m, 1H); ¹³C NMR δ 208.9, 163.3 (d), 141.2 (s), 136.3 (d), 130.7 (dd), 128.8, 128.8, 126.3 (3 d), 125.6 (dd), 116.9, 114.6 (2 dd), 67.3 (d), 50.6, 49.1, 38.4, 32.0 (4 t).

### 6-(3-Fluorophenyl)-4-hydroxy-1-(2-methoxyphenyl)-2-hexanone (22)

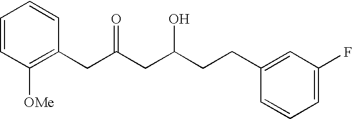

Yield 80%, a colourless viscous oil; ¹H NMR δ 7.28-7.08 (m, 3H), 6.95-6.83 (m, 5H), 4.00 (m, 1H), 3.77 (s, 3H), 3.66 (s, 2H), 3.24 (br s, 1H, OH), 2.75 (m, 1H), 2.67-2.63 (m, 2H), 2.55 (dd, 1H, J=17.6, 8.7), 1.75 (m, 1H), 1.62 (m, 1H); ¹³C NMR δ 162.9 (d), 157.3, 157.3, (2 s), 144.6 (d), 131.2 (d), 129.7 (dd), 128.8 (d), 124.1 (dd), 123.0 (s), 120.8 (d), 115.2, 112.7 (2 dd), 110.6, 66.8 (2 d), 55.3 (q), 48.2, 45.5, 37.7, 31.5 (4 t).

### 5-Hydroxy-1-(4-hydroxy-phenyl)-5-phenyl-hexan-3-one (23)

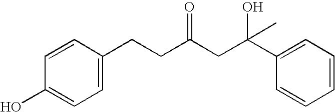

Yield 25%, a yellow viscous oil, ¹H NMR δ 7.37 (m, 2H), 7.30 (m, 2H), 7.21 (m, 1H), 6.87 (m, 2H), 6.68 (m, 2H), 6.05 (br s, 1H, OH), 4.74 (br s, 1H, OH), 3.14 (d, 1H, J=16.8), 2.77 (d, 1H, J=16.8), 2.72-2.59 (m, 3H), 2.53 (m, 1H), 1.49 (s, 3H); ¹³C NMR δ 212.4, 154.2, 146.9, 132.2 (4 s), 129.3, 128.4, 126.9, 124.3, 115.4 (5 d), 73.8 (s), 53.4, 46.2 (2 t), 30.6 (q), 28.3 (t).

### 5-Hydroxy-6-(4-methoxyphenyl)-1-phenyl-hexan-3-one (24)

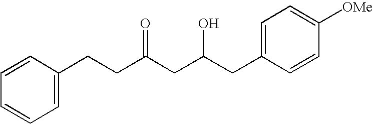

Yield 74%, a yellow wax; ¹H NMR δ 7.26-7.20 (m, 2H), 7.16-7.12 (m, 3H), 7.07 (m, 2H), 6.81 (m, 2H), 4.21 (m, 1H), 3.73 (s, 3H), 2.86-2.82 (m, 2H), 2.75-2.60 (m, 4 H), 2.48 (m, 2H); ¹³C NMR δ 210.5, 158.3, 140.8 (3 s), 130.3 (d), 129.8 (s), 128.5, 128.2, 126.1, 113.9, 68.8 (5 d), 55.2 (q), 48.4, 45.0, 42.1, 29.4 (4 t).

### 5-Hydroxy-6-(4-hydroxy-phenyl)-1-phenyl-hexan-3-one (25)

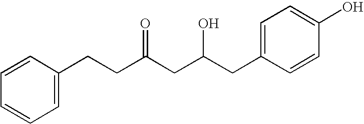

Yield 99%, a brown viscous oil, ¹H NMR δ 7.24 (m, 2H), 7.16 (m, 1H), 7.11 (m, 2H), 6.97 (m, 2H), 6.71 (m, 2H), 4.23 (m, 1H), 2.84 (t, 2H, J=15.0), 2.73-2.68 (m, 3H), 2.61 (dd, 1H, J=13.0, 5.5), 2.52 (m, 2H); ¹³C NMR δ 211.2, 154.8, 140.6 (3 s), 130.5 (d), 129.2 (s), 128.5, 128.2, 126.2, 115.5, 69.1 (5 d), 48.3, 45.0, 42.0, 29.4 (4 t).

### 6-(4-Fluorophenyl)-5-hydroxy-1-phenyl-hexan-3-one (26)

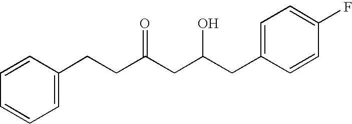

Yield 66%, a yellow viscous oil; ¹H NMR δ 7.25 (m, 2H), 7.17 (m, 1H), 7.15-7.10 (m, 4H), 6.96 (m, 2H), 4.23 (m, 1H), 2.96 (s, 1H, OH), 2.87 (t, 2H, J=7.5), 2.77-2.70 (m, 3H), 2.65 (dd, 1H, J=13.8, 5.9), 2.50 (m, 2H); ¹³C NMR δ 210.5, 161.7 (d), 140.7 (s), 133.6 (d), 130.8 (dd), 128.5, 128.2, 126.2 (3 d), 115.2 (dd), 68.5 (d), 48.4, 45.0, 42.0, 29.4 (4 t).

### 5-Hydroxy-6-naphthalen-1-yl-1-phenyl-hexan-3-one (27)

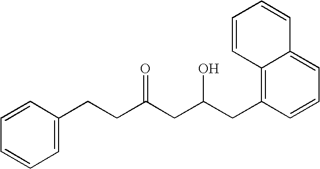

Yield 95%, a pale brown viscous oil; ¹H NMR δ 8.07 (d, 1H, J=8.3), 7.84 (d, 1H, J=7.4), 7.74 (d, 1H, J=8.3), 7.53-7.45 (m, 2H), 7.38 (dd, 1H, J=8.1, 7.0), 7.30 (d, 1H, J=6.6), 7.23 (m, 2H), 7.17-7.10 (m, 3H), 4.44 (m, 1H), 3.26 (dd, 1H, J=13.8, 7.0), 2.92 (s, 1H, OH), 2.84 (m, 2H), 2.74-2.65 (m, 2H), 2.62-2.53 (m, 2H); ¹³C NMR δ 210.5, 140.6, 134.0, 134.0, 132.1 (5 s), 128.8, 128.5, 128.2, 127.7, 127.5, 126.2, 126.1, 125.7, 125.4, 123.9, 68.0 (11 d), 48.7, 45.0, 40.1, 29.4 (4 t).

### 5-Hydroxy-6-naphthalen-2-yl-1-phenyl-hexan-3-one (28)

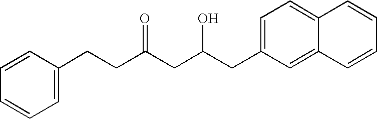

Yield 90%, a yellow wax; ¹H NMR δ 7.79 (d, 1H, J=7.6), 7.77 (d, 1H, J=7.1), 7.77 (d, 1H, J=8.4), 7.61 (s, 1H), 7.44 (m, 2H), 7.31 (dd, 1H, J=8.4, 1.6), 7.23 (m, 2H), 7.15 (m, 1H), 7.12 (m, 2H), 4.37 (m, 1H), 2.96 (dd, 1H, J=13.5, 7.1), 2.88-2.82 (m, 3H), 2.71-2.67 (m, 2H), 2.54 (m, 2H); ¹³C NMR δ 210.5, 140.7, 135.4, 133.5, 132.3 (5 s), 128.5, 128.2, 128.2, 127.9, 127.7, 127.6, 127.5, 126.2, 126.1, 125.5, 68.6 (11 d), 48.4, 45.0, 43.1, 29.4 (4 t).

### 6-(3-Fluorophenyl)-5-hydroxy-1-(2-methoxyphenyl)-hexan-3-one (29)

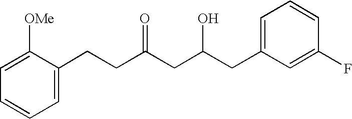

Yield 74%, a colourless viscous oil; ¹H NMR δ 7.28-7.22 (m, 1H), 7.18 (ddd, 1H, J=8.2, 7.4, 1.8), 7.09 (dd, 1H, J=7.4, 1.8), 6.97 (d, 1H, J=8.2), 6.94-6.91 (m, 2H), 6.86 (ddd, 1H, J=7.4, 7.4, 1.1), 6.82 (d, 1H, J=8.2), 4.27 (m, 1H), 3.80 (s, 3H), 3.10 (br s, 1H, OH), 2.90 (m, 2H), 2.82 (dd, 1H, J=13.8, 7.2), 2.72-2.67 (m, 3H), 2.57 (dd, 1H, J=17.6, 3.4), 2.51 (dd, 1H, J=13.8, 8.5); ¹³C NMR δ 211.7 (s), 163.3 (d), 157.6 (s), 140.9 (d), 130.4 (d), 130.3 (dd), 129.3 (s), 128.0 (d), 125.4 (dd), 120.9 (d), 116.7, 113.8 (2 dd), 110.7, 68.8 (2 d), 55.6 (q), 48.5, 43.9, 42.9, 25.2 (4 t).

### 5-Hydroxy-1-(2-hydroxy-phenyl)-6-phenyl-hexan-3-one (30)

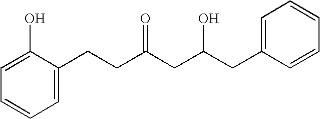

Yield 68%, brown viscous oil, ¹H NMR δ 7.26 (m, 2H), 7.19 (m, 1H), 7.14 (m, 2H), 7.07 (t, 1H, J=7.8), 6.65-6.62 (m, 2H), 6.59 (m, 1H), 4.29 (m, 1H), 2.80 (dd, 1H, J=13.6, 7.2), 2.78-2.73 (m, 2H), 2.68 (dd, 1H, J=13.6, 6.4), 2.66-2.63 (m, 2H), 2.51 (m, 2H); ¹³C NMR δ 211.1, 156.1, 142.4, 13.6 (4 s), 129.7, 129.4, 128.6, 126.6, 120.2, 115.3, 113.3, 68.9 (8 d), 48.3, 44.7, 42.9, 29.2 (4 t).

### 5-Hydroxy-1-(4-hydroxy-phenyl)-6-phenyl-hexan-3-one (31)

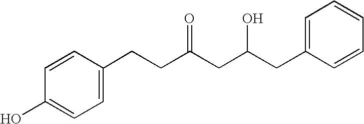

Yield 54%, brown viscous oil, ¹H NMR δ 7.27 (m, 2H), 7.21 (m, 1H), 7.15 (m, 2H) 6.94 (m, 2H), 6.70 (m, 2H), 4.67 (br s, 1H, OH), 4.29 (m, 1H), 2.81 (dd, 1H, J=13.6, 7.2), 2.77-2.73 (m, 2H), 2.69 (dd, 1H, J=13.7, 6.5), 2.66-2.62 (m, 2H), 2.51 (m, 2H); ¹³C NMR δ 211.4, 154.4, 137.6, 132.2 (4 s), 129.4, 129.3, 128.6, 126.6, 115.5, 68.9 (6 d), 48.3, 45.3, 42.9, 25.1 (4 t).

### 6-(4-Fluoro-phenyl)-5-hydroxy-1-(2-hydroxy-phenyl)-hexan-3-one (32)

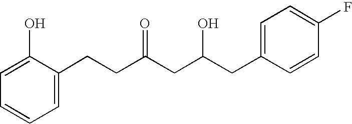

Yield 94%, a brown wax, ¹H NMR δ 7.09-7.03 (m, 3H), 6.91 (m, 2H), 6.64-6.61 (m, 2H), 6.59 (m, 1H), 4.25 (m, 1H), 2.76-2.71 (m, 3H), 2.66-2.60 (m, 3H), 2.50 (dd, 1H, J=17.3, 8.3), 2.47 (dd, 1H, J=17.3, 3.8); ¹³C NMR δ 211.3 (s), 161.7 (d), 156.1, 142.4 (2 s), 133.3 (d), 130.8 (dd), 129.7, 120.2, 115.3 (d), 115.2 (dd), 113.3, 68.9 (2 d), 48.3, 44.7, 41.9, 29.1 (4 t).

### 6-(4-Fluoro-phenyl)-5-hydroxy-1-(4-hydroxy-phenyl)-hexan-3-one (33)

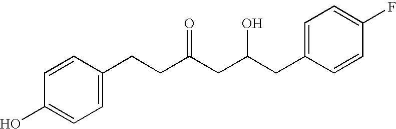

Yield 99%, a dark brown viscous oil, ¹H NMR δ 7.08 (m, 2H), 6.95-6.90 (m, 4H), 6.69 (m, 2H), 4.25 (m, 1H), 2.78-2.72 (m, 3H), 2.67-2.61 (m, 3H), 2.50 (dd, 1H, J=17.3, 7.6), 2.49 (dd, 1H, J=17.3, 4.0); ¹³C NMR δ 211.5 (s), 161.7 (d), 154.4 (s), 133.3 (d), 132.2 (s), 130.8 (dd), 129.3, 115.5 (2 d), 115.3 (dd), 68.9 (d), 48.3, 45.3, 41.9, 28.6 (4 t).

### 1-Hydroxy-6-(4-hydroxy-phenyl)-1-phenyl-hexan-3-one (34)

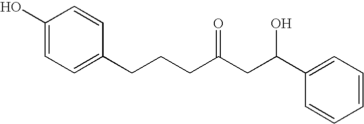

Yield 14%, a brown viscous oil; ¹H NMR δ 7.33 (m, 4H), 7.24 (m, 1H), 6.94 (m, 2H), 6.71 (m, 2H), 6.33 (br s, 1H, OH), 5.12 (dd, 1H, J=9.3, 3.3), 4.00 (br s, 1H, OH), 2.81 (dd, 1H, J=17.2, 9.3), 2.71 (dd, 1H, J=17.2, 3.3), 2.48 (t, 2H, J=7.5), 2.39 (t, 2H, J=7.3), 1.83 (tt, 2H, J=7.5, 7.3); ¹³C NMR δ 211.7, 154.2, 142.6, 133.1 (4 s), 129.5, 128.6, 127.7, 125.7, 115.3, 70.1 (6 d), 50.9, 42.8, 34.0, 25.1 (4 t).

### 1-Hydroxy-1-(3-hydroxy-phenyl)-6-(4-hydroxy-phenyl)-hexan-3-one (35)

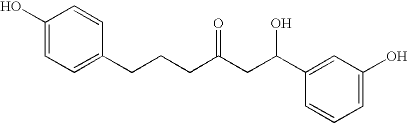

Yield 19%, a brown viscous oil; ¹H NMR δ 8.51 (br s, 1H, OH), 8.21 (br s, 1H, OH), 7.12 (t, 1H, J=7.8), 6.93 (m, 2H), 6.79-6.76 (m, 2H), 6.73-6.69 (m, 3H), 5.01 (dd, 1H, J=9.1, 3.5), 4.37 (br s, 1H, OH), 2.77 (dd, 1H, J=16.8, 9.1), 2.65 (dd, 1H, J=16.8, 3.5), 2.47 (t, 2H, J=7.4), 2.36 (td, 2H, J=7.4, 2.5), 1.80 (tt, 2H, J=7.4, 7.4); ¹³C NMR δ 211.8, 156.7, 154.6, 144.6, 132.7 (5 s), 129.7, 129.5, 117.3, 115.3, 114.9, 112.6, 69.9 (7 d), 51.1, 42.8, 34.0, 25.2 (4 t).

### 6-Hydroxy-1,6-diphenyl-heptan-4-one (36)

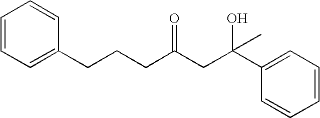

Yield 16%, yellow viscous oil, ¹H NMR δ 7.40 (m, 2H), 7.31 (m, 2H), 7.25 (m, 2H), 7.21 (m, 1H), 7.17 (m, 1H), 7.07 (m, 2H), 3.10 (d, 1H, J=16.8), 2.75 (d, 1H, J=16.8), 2.48 (m, 2H), 2.34 (dt, 1H, J=17.4, 7.3), 2.24 (dt, 1H, J=17.4, 7.3), 1.77 (m, 2H), 1.50 (s, 3H); ¹³C NMR δ 212.6, 147.3, 141.3 (3 s), 128.4, 128.4, 128.3, 126.7, 126.0, 124.3 (6 d), 73.4 (s), 53.3, 46.3, 34.7 (3 t), 30.7 (q), 24.5 (t).

### 6-Hydroxy-1-(4-hydroxy-phenyl)-6-phenyl-heptan-4-one (37)

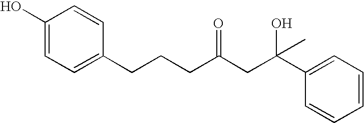

Yield 11%, a brown viscous oil; ¹H NMR δ 7.39 (m, 2H), 7.31 (m, 2H), 7.21 (m, 1H), 6.90 (m, 2H), 6.72 (m, 2H), 5.90 (br s, 1H, OH), 4.73 (br s, 1H, OH), 3.13 (d, 1H, J=16.9), 2.76 (d, 1H, J=16.9), 2.43-2.29 (m, 4H), 1.72 (m, 2H), 1.51 (s, 3H); ¹³C NMR δ 213.1, 154.1, 147.0, 133.1 (4 s), 129.5, 128.3, 126.8, 124.3, 115.3, 73.7 (6 d), 53.2, 43.6, 33.8 (3 t), 30.6 (q), 24.8 (t).

### 2-Hydroxy-1,7-diphenyl-heptan-4-one (38)

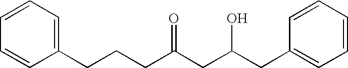

Yield 64%, a colourless oil; ¹H NMR δ 7.31-7.21 (m, 5H), 7.20-7.14 (m, 2H), 7.14-7.11 (m, 3H), 4.25 (m, 1H), 3.02 (br s, 1H, OH), 2.80 (dd, 1H, J=13.6, 7.1), 2.69 (dd, 1H, J=13.6, 7.1), 2.58 (t, 2H, J=7.6), 2.49 (dd, 1H, J=15.7, 2.5), 2.47 (dd, 1H, J=15.7, 6.1), 2.37 (dd, 2H, J=7.9, 6.7), 1.86 (m, 2H); ¹³C NMR δ 211.3, 141.4, 137.9 (3 s), 129.4, 128.5, 128.4, 128.4, 126.5, 126.0, 68.7 (7 d), 48.2, 42.9, 42.7, 34.9, 24.8 (5 t).

### 2-Hydroxy-1-(4-hydroxy-phenyl)-7-phenyl-heptan-4-one (39)

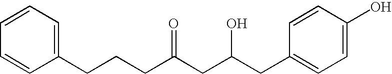

Yield 99%, a pale brown wax; ¹H NMR δ 7.26 (m, 2H), 7.17 (m, 1H), 7.13 (m, 2H), 7.00 (m, 2H), 6.72 (m, 2H), 4.23 (m, 1H), 2.72 (dd, 1H, J=13.7, 7.2), 2.63 (dd, 1H, J=13.7, 6.2), 2.58 (t, 2H, J=7.6), 2.51 (m, 2H), 2.38 (t, 2H, J=7.4), 1.87 (tt, 2H, J=7.6, 7.4); ¹³C NMR δ 212.0, 154.7, 141.3 (3 s), 130.5 (d), 129.3 (s), 128.4, 128.4, 126.0, 115.5, 69.1, (6 d), 48.0, 42.8, 41.9, 34.9, 24.8 (5 t).

### 2-Hydroxy-7-(4-hydroxy-phenyl)-1-phenyl-heptan-4-one (40)

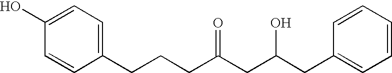

Yield 49%, a pale brown viscous oil; ¹H NMR δ 7.27 (m, 2H), 7.21 (m, 1H), 7.16 (m, 2H), 6.94 (m, 2H), 6.72 (m, 2H), 4.29 (m, 1H), 2.82 (dd, 1H, J=13.6, 8.9), 2.70 (dd, 1H, J=13.6, 6.4), 2.52 (m, 2H), 2.48 (t, 2H, J=7.5), 2.35 (t, 2H, J=7.3), 1.81 (tt, 2H, J=7.5, 7.3); ¹³C NMR δ 212.3, 154.1, 137.6, 133.1 (4 s), 129.5, 129.4, 128.6, 126.6, 115.3, 69.0, (6 d), 48.0, 42.8, 42.7, 34.0, 25.1 (5 t).

### 2-Hydroxy-7-(3,4-dihydroxy-phenyl)-1-phenyl-heptan-4-one (41)

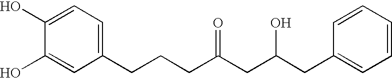

Yield 59%, a reddish brown viscous oil, ¹H NMR δ 7.26-7.21 (m, 2H), 7.18 (m, 1H), 7.13-7.10 (m, 2H), 6.70 (d, 1H, J=7.3), 6.59 (d, 1H, J=1.8), 6.47 (dd, 1H, J=7.3, 1.8), 4.28 (m, 1H), 2.78 (dd, 1H, J=13.5, 7.1), 2.66 (dd, 1H, J=13.5, 6.2), 2.53-2.40 (m, 2H), 2.35 (t, 2H, J=7.0), 2.28 (t, 2H, J=7.0), 1.64 (m, 2H); ¹³C NMR δ 213.1, 144.2, 142.5, 138.0, 134.5 (5 s), 129.8, 129.0, 127.1, 121.0, 116.1, 115.9, 69.5 (7 d), 48.5, 43.2, 43.0, 34.5, 25.3 (5 t).

### 2-Hydroxy-1,7-di-(4-hydroxy-phenyl)-heptan-4-one (42)

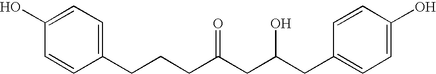

Yield 16%, a colourless viscous oil; ¹H NMR δ 7.06 (m, 2H), 6.99 (m, 2H), 6.76 (m, 2H), 6.73 (m, 2H), 4.22 (m, 1H), 2.95 (br s, 1H, OH), 2.86 (dd, 1H, J=13.6, 7.1), 2.65 (dd, 1H, J=13.6, 6.2), 2.55-2.51 (m, 3H), 2.47 (dd, 1H, J=17.4, 8.5), 2.38 (t, 2H, J=7.3), 1.85 (tt, 2H, 7.5, 7.3); ¹³C NMR δ 215.9, 154.3, 153.8, 133.6 (4 s), 130.6, 129.5 (2d), 125.0 (s), 115.4, 115.3, 68.9 (3 d), 48.1, 42.6, 42.0, 29.7, 25.1 (5 t).

### 6-Hydroxy-1,8-diphenyl-octan-4-one (43)

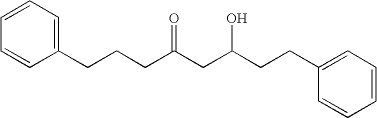

Yield 78%, yellow viscous oil, ¹H NMR δ 7.27-7.22 (m, 4H), 7.20-7.14 (m, 6H), 4.03 (m, 1H), 2.81 (m, 1H), 2.68 (m, 1H), 2.61 (t, 2H, J=7.5), 2.53 (m, 2H), 2.41 (t, 2H, J=7.4), 1.91 (tt, 2H, J=7.4), 1.81 (m, 1H), 1.67 (m, 1H); ¹³C NMR δ 211.7, 141.8, 141.3 (3 s), 128.5, 128.4, 128.4, 128.4, 126.0, 125.8, 66.9 (7 d), 49.1, 42.7, 38.1, 35.0, 31.7, 24.9 (6 t).

### Examples 44-52

### 4-Methoxy-1,6-diphenyl-hexan-2-one (44)

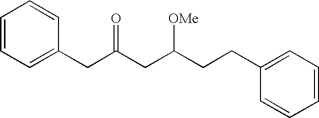

Method B: To a stirred solution of 4-hydroxy-1,6-diphenylhexan-2-one (compound 20, 0.27 g, 0.001 mmol) in 25 ml of methanol is added dropwise concentrated HCl (0.2 ml) at 0° C., and the mixture is refluxed overnight. Then the solution is taken up with dichloromethane, washed with saturated NaHCO₃ and water, dried with MgSO₄ and evaporated to dryness. The residue is purified by column chromatography using dichloromethane as an eluent.

Yield 40%, a pale brown oil; ¹H NMR δ 7.31-7.21 (m, 5H), 7.17-7.11 (m, 5H), 3.67 (m, 1H), 3.66 (s, 2H), 3.27 (s, 3H), 2.71 (dd, 1H, J=16.0, 6.9), 2.61 (m, 2H), 2.49 (dd, 1H, J=16.0, 5.4), 1.75 (m, 2H); ¹³C NMR δ 206.7, 141.8, 133.9 (3 s), 129.5, 128.7, 128.3, 128.3, 127.0, 125.8, 76.5 (7 d), 56.9 (q), 51.0, 46.3, 35.6, 31.2 (4 t).

The following compounds included in the invention were prepared by Method B using appropriate starting materials:

### 4-Methoxy-1,4-diphenyl-butan-2-one (45)

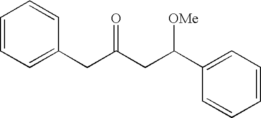

Yield 20%, a pale yellow oil, ¹H NMR δ 7.35-7.20 (m, 6H), 7.16-7.10 (m, 4H), 4.64 (dd, 1H, J=8.8, 4.5), 3.69 (d, 1H, J=15.8), 3.65 (d, 1H, J=15.8), 3.18 (s, 3H), 2.98 (dd, 1H, J=15.9, 8.8), 2.59 (dd, 1H, J=15.9, 4.5; ¹³C NMR δ 205.6, 141.0, 133.8 (3 s), 129.5, 128.6, 128.5, 127.8, 127.0, 126.5, 79.6 (7 d), 56.7 (q), 51.0, 50.3 (2 t).

### 5-Methoxy-1,6-diphenyl-hexan-3-one (46)

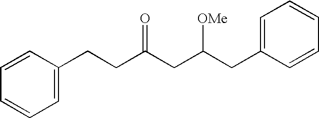

Yield 57%, a brown oil; ¹H NMR δ 7.26-7.20 (m, 4H), 7.19-7.10 (m, 6H), 3.90 (m, 1H), 3.28 (s, 3H), 2.89-2.80 (m, 3H), 2.76-2.61 (m, 3H), 2.56 (dd, 1H, J=16.1, 7.9) 2.36 (dd, 1H, J=16.1, 4.5); ¹³C NMR δ 208.3, 141.0, 137.9 (3 s), 129.7, 128.4, 128.4, 128.3, 126.4, 126.0, 78.6 (7 d), 57.3 (q), 47.0, 45.3, 39.8, 29.4 (4 t).

### 5-Methoxy-6-(4-methoxyphenyl)-1-phenyl-hexan-3-one (47)

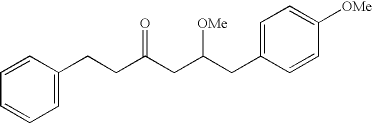

Yield 42%, a reddish brown oil; ¹H NMR δ 7.24-7.21 (m, 2H), 7.20-7.12 (m, 3H), 7.07 (m, 2H), 6.81 (m, 2H), 3.86 (m, 1H), 3.77 (s, 3H), 3.29 (s, 3H), 2.86-2.79 (m, 3H), 2.73-2.62 (m, 3H), 2.57 (dd, 1H, J=16.1, 7.9), 2.39 (dd, 1H, J=16.1, 4.5); ¹³C NMR δ 208.5, 158.2, 141.1 (3 s), 130.4 (d), 129.9 (s), 128.4, 128.3, 126.0, 113.8, 78.5 (5 d), 57.3, 55.2 (2 q), 47.0, 45.4, 38.9, 29.4 (4 t).

### 5-Methoxy-6-(4-fluorophenyl)-1-phenyl-hexan-3-one (48)

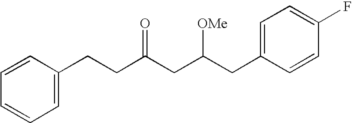

Yield 89%, a yellow viscous oil; ¹H NMR δ 7.26 (m, 2H), 7.18-7.14 (m, 3H), 7.11 (m, 2H), 6.95 (m, 2H), 3.86 (m, 1H), 3.27 (s, 3H), 2.86 (t, 2H, J=7.7), 2.79 (dd, 1 H, J=13.9, 6.0), 2.76-2.65 (m, 3H), 2.57 (dd, 1H, J=16.2, 7.6) 2.36 (dd, 1H, J=16.2, 4.8); ¹³C NMR δ 208.3, 161.6 (d), 141.0 (s), 133.6 (d), 130.9 (dd), 128.5, 128.3, 126.1 (3 d), 115.1 (dd), 78.2 (d), 57.4 (q), 46.9, 45.4, 39.0, 29.4 (4 t).

### 5-Methoxy-6-naphthalen-1-yl-1-phenyl-hexan-3-one (49)

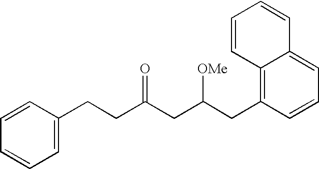

Yield 83%, a pale brown viscous oil; ¹H NMR δ 8.08 (d, 1H, J=8.5), 7.81 (d, 1H, J=8.1), 7.70 (d, 1H, J=8.1), 7.51 (dd, 1H, J=8.1, 7.0), 7.45 (dd, 1H, J=8.1, 7.0), 7.36 (dd, 1H, J=7.1, 7.1), 7.31 (d, 1H, J=6.7), 7.21 (m, 2H), 7.15-7.08 (m, 3H), 4.09 (m, 1H), 3.36 (dd, 1H, J=13.9, 6.4), 3.24 (s, 3H), 3.10 (dd, 1H, J=13.9, 6.8), 2.80 (m, 2H), 2.70-2.57 (m, 3H), 2.40 (dd, 1H, J=16.2, 4.6); ¹³C NMR δ 208.3, 141.0, 134.2, 133.9, 132.2 (5 s), 128.8, 128.4, 128.3, 127.9, 127.3, 126.0, 126.0, 125.5, 125.4, 123.8, 77.8 (11 d), 57.6 (q), 47.6, 45.3, 37.9, 29.4 (4 t).

### 5-Methoxy-6-naphthalen-2-yl-1-phenyl-hexan-3-one (50)

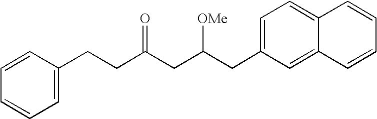

Yield 95%, a pale brown viscous oil; ¹H NMR δ 7.80 (d, 1H, J=7.3), 7.77 (d, 1H, J=7.1), 7.76 (d, 1H, J=8.3), 7.61 (s, 1H), 7.44 (m, 2H), 7.32 (dd, 1H, J=8.3, 1.7), 7.23 (m, 2H), 7.14 (m, 1H), 7.11 (m, 2H), 4.01 (m, 1H), 3.32 (s, 3H), 3.03 (dd, 1H, J=13.7, 5.9), 2.88-2.79 (m, 3H), 2.74-2.59 (m, 3H), 2.42 (dd, 1H, J=16.1, 4.6); ¹³C NMR δ 208.4, 141.0, 135.6, 133.5, 132.2 (5 s), 128.4, 128.3, 128.0, 128.0, 127.9, 127.6, 127.5, 126.0, 126.0, 125.5, 78.3 (11 d), 57.5 (q), 47.1, 45.4, 40.1, 29.4 (4 t).

### 2-Methoxy-1,7-diphenyl-heptan-4-one (51)

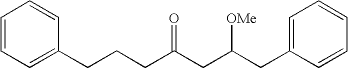

Yield 36%, a reddish brown oil; ¹H NMR δ 7.30-7.23 (m, 4H), 7.21-7.11 (m, 6H), 3.91 (m, 1H), 3.29 (s, 3H), 2.86 (dd, 1H, J=13.7, 5.8), 2.70 (dd, 1H, J=13.7, 6.6), 2.61-2.50 (m, 3H), 2.42-2.29 (m, 3H), 1.85 (m, 2H); ¹³C NMR δ 209.1, 141.6, 138.0 (3 s), 129.5, 128.4, 128.4, 128.3, 126.3, 125.9, 78.3 (7 d), 57.3 (q), 46.9, 43.0, 39.8, 35.0, 24.9 (5 t).

### 3-Methoxy-1,8-diphenyl-octan-5-one (52)

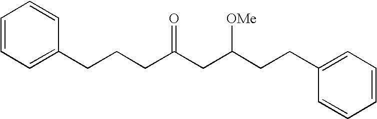

Yield 56%, colourless viscous oil, ¹H NMR δ 7.26 (m, 2H), 7.19-7.14 (m, 3H), 3.70 (m, 1H), 3.31 (s, 3H), 2.70-2.58 (m, 5H), 2.42 (m, 3H), 1.90 (m, 2H), 1.79 (m, 2H); ¹³C NMR δ 209.3, 141.9, 141.6 (3 s), 128.5, 128.4, 128.4, 128.3, 125.9, 125.9 (6 d), 76.7 (d), 57.0 (q), 47.2, 43.0, 35.7, 35.0, 31.4, 25.0 (6 t).

### Chiral Separations of the Racemic Mixtures.

The enantiomers were separated by semi preparative HPLC using a Regis Technologies (R,R)-Whelk-O 1 (25 cm×10 mm i.d.) chiral stationary phase in Hexane/i-PrOH/AcOH 80/20/0.5 at flow rate 5 mL/min.

### Determination of the Estrogen Activity of the Compounds: Cell Culture, Transfection and Reporter Assays.

E2 was bought from Sigma Chemical Co. (St. Louis, Mo.), and ICI-182,780 was from Tocris (Avonmouth, UK). All other reagents were of reagent grade from Sigma or Fluka. One day before transfection, HEK293 cells were seeded in 48-well plates (70×10³ cells per well) in phenol-free Dulbecco's modified Eagle medium supplemented with 5% delipidated fetal bovine serum (Sigma) and antibiotics. After a medium change, the cells were transfected for 4 hours with 5 ng ERα or ERβ expression vector, 75 ng reporter plasmid pERE₂TATA-LUC and 20 ng control plasmid pCMVβ by the calcium phosphate method. After transfection, the cells received fresh medium containing either vehicle (0.1% v/v) or test compound (10 μM). After 24 hours, the cells were washed, lysed and luciferase and β-galactosidase activities were determined from 20 μl of lysates with the Victor^{2TM} reader (PerkinElmer Wallac, Turku, Finland). After normalization for β-galactosidase activity, luciferase activities are expressed relative to that of 10 nM E2 by the formula: Activity=100%×[(Test compound)−(Vehicle)/(E2)−(Vehicle)], where terms in parenthesis indicate the corresponding normalized luciferase activities. Typically, more than 90-fold activation by 10 nM E2 of luciferase with both ER subtypes was seen. The data are means±SEM of at least three independent transfections.

The in vitro estrogen activity of the compounds of the invention is presented in Table 1.

TABLE 1

| No. | ERα^{a} | ERβ^{a} |
|---|---|---|
| 12ab^{b} | 0 | −8.7 |
| 18ab | 22.6 | 58.0 |
| 19ab | 12.0 | 7.7 |
| 20ab | 4.3 | 0.2 |
| 21ab | 25.1 | 21.1 |
| 22a^{c} | 8.4 | 3.4 |
| 22b^{d} | 5.6 | 2.9 |
| 23ab | 80.3 | 116.3 |
| 24a | 1.6 | −0.8 |
| 24b | 1.3 | 1.2 |
| 25ab | 51.4 | 6.1 |
| 26ab | 35.0 | 3.9 |
| 27ab | 12.1 | 1.7 |
| 28a | 12.4 | 6.4 |
| 28b | 20.1 | 20.1 |
| 30ab | 45.2 | 5.1 |
| 31ab | 55.0 | 106.7 |
| 32ab | 21.6 | 11.1 |
| 33ab | 28.6 | 21.2 |
| 34a | 139.2 | 170.2 |
| 34b | 140.0 | 155.3 |
| No. | ERα | ERβ |
| 35a | 87.4 | 107.8 |
| 35b | 49.3 | 43.8 |
| 36ab | 83.3 | 15.1 |
| 37ab | 90.9 | 103.1 |
| 38ab | 24.7 | 9.1 |
| 39ab | 16.0 | 2.4 |
| 40ab | 105.2 | 82.3 |
| 41ab | −0.5 | −0.8 |
| 42a | 15.4 | 1.8 |
| 42b | 2.8 | −1.6 |
| 43ab | 2.2 | −7.1 |
| 44ab | 12.0 | 0.2 |
| 46ab | 9.9 | 1.3 |
| 47ab | 28.5 | 4.0 |
| 48ab | 33.1 | 11.2 |
| 49ab | 35.5 | 2.0 |
| 50ab | 30.4 | 12.4 |
| 51ab | 24.5 | 30.4 |
| 52ab | 3.1 | −14.6 |

^{a}Receptor activation (a mean of at least three independent transfections, SEM typically <15%) relative to 10 nM E2 corresponding 100, sample concentration 10 μM.
^{b}ab = enantiomers not separated, rasemic mixture tested.
^{c}a = enantiomer with a shorter retention time in the chiral separation.
^{d}b = enantiomer with a longer retention time in the chiral separation.

The in vitro activity of the compounds which are disclosed for the first time for a medical use in the present application is presented in Table 2.

TABLE 2
Previously published compounds

| No. | ERα | ERβ |
|---|---|---|
| 53ab | 5.3 | 16.9 |
| 54a | 3.0 | 1.1 |
| 54b | 30.2 | 7.3 |
| 55ab | 0.8 | 1.6 |
| 56a | 78.8 | 20.5 |
| 56b | 25.3 | 2.8 |
| 57ab | 52.1 | 38.4 |
| 58ab | 12.8 | 0.4 |
| 59a | 91.4 | 147.3 |
| 59b | 59.3 | 28.2 |
| 60ab | 83.3 | 15.1 |
| 61ab | 0.3 | 18.4 |
| 62ab | 38.1 | 28.8 |

Compounds 53-60 were synthesized according to method A.

### 3-Hydroxy-1,4-diphenyl-butan-1-one (53)

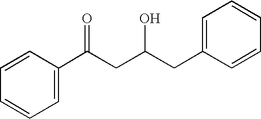

Yield 28%, mp=85.0-86.0° C., ¹H NMR δ 7.90 (m, 2H), 7.59-7.42 (m, 4H), 7.34-7.20 (m, 6H), 4.48 (m, 1H), 3.30 (d, 1H, OH, J=3.5), 3.12 (dd, 1H, J=17.6, 3.4), 3.08 (dd, 1H, J=17.6, 8.3), 2.96 (dd, 1H, J=13.6, 7.1), 2.84 (dd, 1H, J=13.6, 6.3), ¹³C NMR δ 200.5, 138.0, 136.7 (3 s), 133.5, 129.5, 128.7, 128.6, 128.1, 126.6, 68.9 (7 d), 44.1, 42.9 (2 t).

### 3-Hydroxy-1,5-diphenyl-pentan-1-one (54)

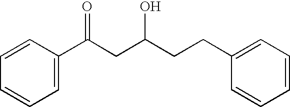

Yield 52%, a pale yellow wax; ¹H NMR δ 7.91 (m, 2H), 7.55 (m, 1H), 7.44 (m, 2H), 7.27 (m, 2H), 7.22 (m, 2H), 7.17 (m, 1H), 4.23 (m, 1H), 3.39 (d, 1H, OH, J=3.2), 3.11 (dd, 1H, J=17.7, 3.0), 3.06 (dd, 1H, J=17.7, 8.7), 2.87 (ddd, 1H, J=14.7, 9.8, 5.4), 2.74 (ddd, 1H, J=14.7, 9.5, 6.9), 1.93 (m, 1H), 1.80 (m, 1H); ¹³C NMR δ 200.8, 141.9, 136.7 (3 s), 133.5, 128.7, 128.5, 128.4, 128.0, 125.8, 67.0 (7 d), 45.1, 38.2, 31.7 (3 t).

### 4-Hydroxy-1,4-diphenyl-butan-2-one (55)

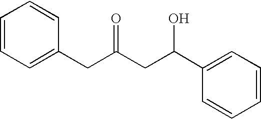

Yield 58%, yellow wax, ¹H NMR δ 7.32-7.27 (m, 5H), 7.27-7.22 (m, 3H), 7.14 (m, 2H), 5.09 (dd, 1H, J=9.1, 3.2), 3.67 (d, 1H, J=15.4), 3.66 (d, 1H, J=15.4), 3.26 (br s, 1H, OH), 2.85 (dd, 1H, J=17.3, 9.1), 2.78 (dd, 1H, J=17.3, 3.2), ¹³C NMR δ 208.5, 142.8, 133.5 (3 s), 129.4, 128.8, 128.5, 127.6, 127.2, 125.6, 69.9 (7 d), 50.8, 50.3 (2 t).

### 1-Hydroxy-1,5-diphenyl-pentan-3-one (56)

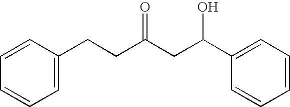

Yield 62%, a colourless oil; ¹H NMR δ 7.28 (m, 4H), 7.23 (m, 3H), 7.15 (m, 1H), 7.11 (m, 2H), 5.08 (dd, 1H, J=9.3, 3.3), 2.85 (t, 2H, J=7.6), 2.78 (dd, 1H, J=17.0, 9.3), 2.68 (m, 2H), 2.66 (dd, 1H, J=17.0, 3.3); ¹³C NMR δ 209.9, 143.0, 140.6 (3 s), 128.4, 128.4, 128.2, 127.5, 126.1, 125.5, 69.8 (7 d), 51.3, 45.0, 29.3 (3 t).

### 5-Hydroxy-1,5-diphenyl-hexan-3-one (57)

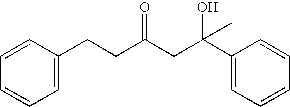

Yield 73%, a pale brown viscous oil, ¹H NMR δ 7.38 (m, 2H), 7.30 (m, 2H), 7.25-7.19 (m3H), 7.15 (m, 1H), 7.04 (m, 2H), 3.11 (d, 1H, J=16.7), 2.77 (d, 1H, J=16.7), 2.77-2.63 (m, 3H), 2.55 (m, 1H), 1.49 (s, 3H); ¹³C NMR δ 211.7, 147.2, 140.5 (3 s), 128.5, 128.3, 128.2, 126.7, 126.1, 124.3 (6 d), 73.3 (s), 53.5, 45.9 (2 t), 30.6 (q), 29.1 (t).

### 5-Hydroxy-1,6-diphenyl-hexan-3-one (58)

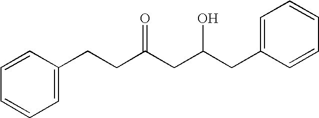

Yield 76%, a yellow wax; ¹H NMR δ 7.28-7.18 (m, 6H), 7.16-7.10 (m, 4H), 4.25 (m, 1H), 3.02 (br s, 1H, OH), 2.83 (t, 2H, J=7.6), 2.77 (dd, 1H, J=13.6, 7.1), 2.69-2.64 (m, 3H), 2.47 (d, 2H, J=6.0); ¹³C NMR δ 210.3, 140.7, 137.9 (3 s), 129.4, 128.5, 128.5, 128.2, 126.5, 126.1, 68.6 (7 d), 48.4, 44.9, 43.0, 29.4 (4 t).

### 1-Hydroxy-1,6-diphenyl-hexan-3-one (59)

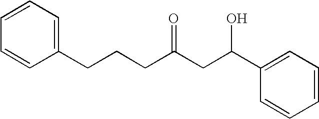

Yield 73%, a pale brown wax; ¹H NMR δ 7.36-7.33 (m, 3H), 7.30-7.25 (m, 3H), 7.19 (m, 1H), 7.15 (m, 2H), 5.13 (dd, 1H, J=9.1, 3.0), 3.21 (br s, 1H, OH), 2.81 (dd, 1H, J=17.5, 9.1), 2.76 (dd, 1H, J=17.5, 3.0), 2.61 (t, 2H, J=7.5), 2.43 (t, 2H, J=7.3), 1.92 (tt, 2H, J=7.5, 7.3); ¹³C NMR δ 210.8, 143.0, 141.5 (3 s), 128.4, 128.4, 128.3, 127.6, 126.0, 125.6, 69.9 (7 d), 51.1, 42.7, 34.9, 24.9 (4 t).

### 6-Hydroxy-1,6-diphenyl-heptan-4-one (60)

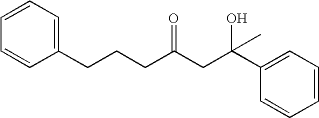

Yield 16%, yellow viscous oil, ¹H NMR δ 7.40 (m, 2H), 7.31 (m, 2H), 7.25 (m, 2H), 7.21 (m, 1H), 7.17 (m, 1H), 7.07 (m, 2H), 3.10 (d, 1H, J=16.8), 2.75 (d, 1H, J=16.8), 2.48 (m, 2H), 2.34 (dt, 1H, J=17.4, 7.3), 2.24 (dt, 1H, J=17.4, 7.3), 1.77 (m, 2H), 1.50 (s, 3H); ¹³C NMR δ 212.6, 147.3, 141.3 (3 s), 128.4, 128.4, 128.3, 126.7, 126.0, 124.3 (6 d), 73.4 (s), 53.3, 46.3, 34.7 (3 t), 30.7 (q), 24.5 (t).

Compounds 61 and 62 were synthesized according to method B.

### 3-Methoxy-1,4-diphenyl-butan-1-one (61)

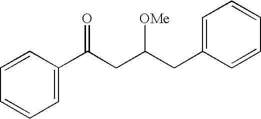

Yield 31%, pale brown oil, ¹H NMR δ 7.89 (m, 2H), 7.54 (m, 1H), 7.43 (m, 2H), 7.29 (m, 2H), 7.25-7.19 (m, 3H), 4.13 (m, 1H), 3.33 (s, 3H), 3.23 (dd, 2H, J=16.5, 7.3), 2.96 (dd, 2H, J=13.8, 6.1), 2.92 (dd, 2H, J=16.5, 4.9), 2.87 (dd, 2H, J=13.8, 6.2); ¹³C NMR δ 198.8, 138.2, 137.3 (3 s), 133.1, 129.6, 128.5, 128.4, 128.1, 126.4, 78.6 (7 d), 57.6 (q), 42.9, 40.2 (2 t).

### 3-Methoxy-1,5-diphenyl-pentan-1-one (62)

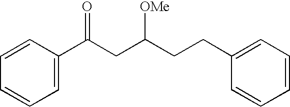

Yield 36%, yellow viscous oil, ¹H NMR δ 7.95 (m, 2H), 7.56 (m, 1H), 7.46 (m, 2H), 7.27 (m, 2H), 7.21-7.15 (m, 3H), 3.93 (m, 1H), 3.37 (s, 3H), 3.32 (dd, 2H, J=16.2, 6.6), 2.98 (dd, 2H, J=16.2, 5.7), 2.74 (m, 2H), 1.91 (m, 2H); ¹³C NMR δ 198.8, 142.0, 137.3 (3 s), 133.1, 128.6, 128.4, 128.4, 128.2, 125.8, 77.1 (7 d), 57.3 (q), 43.2, 36.2, 31.5 (3 t).

### Determination of Relative Binding Affinity (RBA).

Relative binding affinities (RBA) were measured by a competitive assay against [6, 7-³H(N)]estradiol (PerkinElmer) in transsiently transfected COS-1 cells. One day before transfection, COS-1 cells were seeded into 2 ml of DMEM (Dulbecco's modified Eagle medium, Gipco) with 10% delipidated fetal bovine serum and 0.25% (vol/vol) Penicillin-Streptomysin (Euroclone) at a density of 140×10³ cells/well. After medium change to DMEM 2.5% FBS, the cells were transfected for 24 hours with 10 ng/well of human ERα/ERβ expression vector pSG5-hERα/β by using the TransIT method (Micrus Bio TransIT-LT1, Transfection Reagent). After 36 hours, the cells where treated with tested compounds using 0.01-, 0.1-, 1-, 10-, 100-, 1000- and 10000-fold molarities compared to labeled E2 (1.96 pM/well). After 2 hours of incubation at 37° C. the medium was removed. The cells were removed from the wells to 150 μl of 1× phosphate buffered saline (PBS), transferred to Eppendorf tubes and centrifuged at 4° C. using 4000 g for 5 min, and then washed twice with 150 μl of PBS. The cell pellets were dissolved to 50 μl of 0.5M NaOH and incubated for 15 min at 56° C., after which the samples were transferred to liquid twinkle tubes and treated with 3 ml of OptiPhase HiSafe 3 twinkle solution (PerkinElmer). The results were measured with LKB WALLAC 1214 racbeta equipment.

TABLE 3
The median inhibition concentration (IC₅₀) and relative
binding affinity (RBA) of some of the estrogen active
compounds in comparison to E2 and tamoxifen (ER antagonist
used for the treatment of breast cancer).

| | ERα | | ERβ | |
|---|---|---|---|---|
| No. | IC₅₀ (M) | RBA (%) | IC₅₀ (M) | RBA (%) |
| E2 | 1.96 × 10⁻⁹ | 100.0 | 1.96 × 10⁻⁹ | 100.0 |
| tamoxifen | 1.30 × 10⁻⁷ | 1.511 | 9.00 × 10⁻⁸ | 2.178 |
| 31ab | 7.90 × 10⁻⁶ | 0.025 | 1.25 × 10⁻⁶ | 0.157 |
| 34a | 9.00 × 10⁻¹⁰ | 217.8 | 3.40 × 10⁻⁹ | 57.65 |
| 34b | 2.13 × 10⁻⁹ | 92.02 | 1.05 × 10⁻⁸ | 18.67 |
| 36ab | 8.10 × 10⁻⁷ | 0.242 | 2.05 × 10⁻⁶ | 0.096 |
| 40ab | 1.05 × 10⁻⁷ | 1.867 | 1.08 × 10⁻⁶ | 0.181 |
| 59a | 1.70 × 10⁻⁶ | 0.115 | 1.09 × 10⁻⁶ | 0.180 |
| 59b | 2.17 × 10⁻⁶ | 0.090 | 1.10 × 10⁻⁵ | 0.018 |

1.-18. (canceled)

19. Compounds of the formula (I) 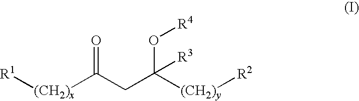
or stereoisomers, pharmaceutically acceptable salts or prodrug forms thereof, wherein
x is an integer from 0 to 3 and y is an integer from 0 to 2, provided that x and y do not have the same value;
R¹ and R² are the same or different phenyl or naphthyl groups of the formula 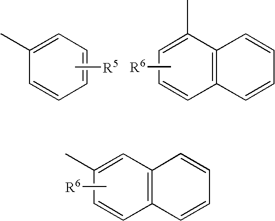
and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl;
provided that
when the compound has the formula (I) and x is 0, y is 1, R³ is hydrogen and R⁴ is hydrogen, then R¹ cannot be an unsubstituted phenyl, 2-hydroxyphenyl or 3-methoxyphenyl if R² is unsubstituted phenyl, and then R¹ cannot be 4-chlorophenyl if R² also is 4-chlorophenyl [a)-d)], and
when the compound has the formula (I) and x is 0, y is 2, R³ is hydrogen and R⁴ is hydrogen, then R¹ cannot be an unsubstituted phenyl, 2-hydroxyphenyl, 4-hydroxyphenyl, 4-methoxyphenyl, 3-nitrophenyl, 4-chlorophenyl or naphthalen-2-yl, if R² is an unsubstituted phenyl, [e)-h)] and
when the compound has the formula (I) and x is 1, y is 0, R³ is hydrogen and R⁴ is hydrogen, then R¹ cannot be an unsubstituted phenyl or 3-fluorophenyl if R² is unsubstituted phenyl, and then R¹ cannot be an unsubstituted phenyl or 3,4-dimethoxyphenyl if R² is 4-methoxyphenyl, and then R¹ cannot be 2-chlorophenyl if R² is 2-chlorophenyl, [i)-1)] and
when the compound has the formula (I) and x is 2, y is 0, and R⁴ is hydrogen, then R³ cannot be hydrogen if R¹ is an unsubstituted phenyl and R² is an unsubstituted phenyl or 4-nitrophenyl, or if R¹ is 4-hydroxy-3-methoxyphenyl and R² is an unsubstituted phenyl or 4-hydroxy-3-methoxyphenyl, and then R³ cannot be methyl, if R¹ and R² are simultaneously unsubstituted phenyls, [m)-q)] and
when the compound has the formula (I) and x is 2, y is 1, R³ is hydrogen and R⁴ is hydrogen, then R¹ cannot be an unsubstituted phenyl if R² also is an unsubstituted phenyl [r)], and
when the compound has formula (I) and x is 3, y is 0, R³ is hydrogen or methyl and R⁴ is hydrogen, then R¹ cannot be an unsubstituted phenyl if R² also is an unsubstituted phenyl, [s)-t)] and
when the compound has the formula (I) and x is 0, y is 1 or 2, R³ is hydrogen and R⁴ is methyl, then R¹ cannot be an unsubstituted phenyl if R² also is an unsubstituted phenyl [u)-v)].

20. The compounds according to claim 19 having formula (I) or stereoisomers, pharmaceutically acceptable salts or prodrug forms thereof, wherein
x is 2 or 3 and y is 0 or 1, R¹ and R² are both phenyl groups which are independently substituted by one substituent selected from the group consisting of alkoxy, halogen or hydroxyl, R³ is hydrogen or methyl and R⁴ is hydrogen or methyl.

21. A compound selected from the group consisting of
5-hydroxy-1-(4-hydroxy-phenyl)-5-phenyl-hexan-3-one (23),
5-hydroxy-6-(4-hydroxy-phenyl)-1-phenyl-hexan-3-one (25),
5-hydroxy-1-(4-hydroxy-phenyl)-6-phenyl-hexan-3-one (31),
1-hydroxy-6-(4-hydroxy-phenyl)-1-phenyl-hexan-3-one (34),
1-hydroxy-1-(3-hydroxy-phenyl)-6-(4-hydroxy-phenyl)-hexan-3-one (35),
6-hydroxy-1,6-diphenyl-heptan-4-one (36),
6-hydroxy-1-(4-hydroxy-phenyl)-6-phenyl-heptan-4-one (37),
2-hydroxy-7-(4-hydroxy-phenyl)-1-phenyl-heptan-4-one (40),
5-methoxy-6-(4-fluorophenyl)-1-phenyl-hexan-3-one (48),
5-methoxy-6-naphthalen-2-yl-1-phenyl-hexan-3-one (50),
2-methoxy-1,7-diphenyl-heptan-4-one (51),
and stereoisomers, pharmaceutically acceptable salts and prodrug forms thereof.

22. A compound according to claim 19 or 21 or a stereoisomer, pharmaceutically acceptable salt or prodrug form thereof for use as a pharmaceutical.

23. A process for preparing the compounds of claim 19, comprising
catalytic hydrogenation of appropriate 4,5-dihydroisoxazoles in the presence of Raney-Ni, acetic acid and water to afford the desired β-hydroxyketones, or
methylation of the corresponding β-hydroxyketones in the presence of methanol and hydrochloric acid to afford the desired β-methoxyketones.

24. A pharmaceutical composition comprising a compound according to claim 19 or a stereoisomer, pharmaceutically acceptable salt or prodrug form thereof in association with a pharmaceutically acceptable carrier.

25. A method for the therapeutic or prophylactic treatment of disease states, disorders or conditions alleviated by compounds having estrogen activity, said method comprising administering an effective amount of a compound according to claim 19 having formula (I) or a stereoisomer, pharmaceutically acceptable salt or prodrug form thereof to a subject in need of such treatment.

26. The method according to claim 25 for the therapeutic or prophylactic treatment of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.

27. Compounds of the formula (I) 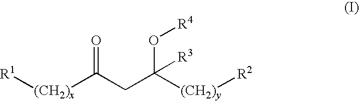
or stereoisomers, pharmaceutically acceptable salts or prodrug forms thereof, wherein
x is an integer from 0 to 3 and y is an integer from 0 to 2, provided that x and y do not have the same value;
R¹ and R² are the same or different phenyl or naphthyl groups of the formula 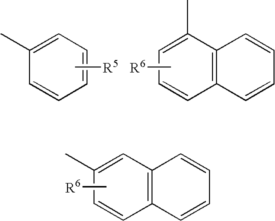
and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl,
for use as pharmaceuticals.

28. The compounds having formula (I) according to claim 27 or stereoisomers, pharmaceutically acceptable salts or prodrug forms thereof for use as pharmaceutical, wherein in the formula (I) x is 2 or 3 and y is 0 or 1, R¹ and R² are both phenyl groups which are independently substituted by one substituent selected from the group consisting of alkoxy, halogen or hydroxyl, R³ is hydrogen or methyl and R⁴ is hydrogen or methyl.

29. A pharmaceutical composition comprising a compound of the formula (I) 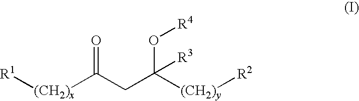
or a stereoisomer, pharmaceutically acceptable salt or a prodrug form thereof; wherein
x is an integer from 0 to 3 and y is an integer from 0 to 2, provided that x and y do not have the same value;
R¹ and R² are the same or different phenyl or naphthyl groups of the formula 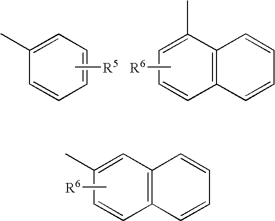
and are substituted with 0-5 R⁵ or R⁶;
R⁵ or R⁶ are the same or different and are selected from hydrogen, lower alkyl, lower alkoxyl, halogen, nitro, amino or hydroxyl;
R³ is selected from hydrogen or C₁₋₄alkyl;
R⁴ is selected from hydrogen or methyl;
in association with a pharmaceutically acceptable carrier.

30. A method for the therapeutic or prophylactic treatment of disease states, disorders or conditions alleviated by compounds having estrogen activity, said method comprising administering an effective amount of a compound having formula (I) according to claim 27 or a stereoisomer, pharmaceutically acceptable salt or prodrug form thereof to a subject in need of such treatment.

31. The method according to claim 30, wherein the disease states, disorders and conditions are selected from the group consisting of bone loss, bone fractures, osteoporosis, metastatic bone disease, Paget's disease, periodontal disease, cartilage degeneration, endometriosis, uterine fibroid disease, hot flashes, anovulation, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression resulting from an estrogen deficiency, inflammation, inflammatory bowel disease, sexual dysfunction, hypertension, retinal degeneration and cancer, in particular of the breast, uterus and prostate.
